(19) United States
(12) Patent Application Publication
(10) Pub. No.: US 2022/0257559 A1
(43) Pub. Date: Aug. 18, 2022

(54) INTESTINAL MICROBIOME-IMPROVING COMPOSITION INCLUDING ELLAGIC ACID AS ACTIVE INGREDIENT

(71) Applicant: Andong National University Industry-Academic Cooperation Foundation, Andong-si, Gyeongsangbuk-do (KR)

(72) Inventors: Young-Eun CHO, Daejeon (KR); Dong-Ha KIM, Gumi-si (KR)

(73) Assignee: Andong National University Industry-Academic Cooperation Foundation, Andong-si, Gyeongsangbuk-do (KR)

(21) Appl. No.: 17/592,478

(22) Filed: Feb. 3, 2022

(30) Foreign Application Priority Data
Feb. 15, 2021 (KR) 10-2021-0019977

(51) Int. Cl. A61K 31/352; A61P 1/16

(52) U.S. Cl. CPC A61K 31/352; A61P 1/16

(57) ABSTRACT

Proposed is an intestinal microbiome-improving composition containing ellagic acid as an active ingredient. The ellagic acid inhibits the growth of harmful bacteria such as Verrucomicrobia and Bacteroidetes attributable to alcohol intake and increases the growth of beneficial bacteria such as Firmicutes, thereby controlling intestinal microbes. As a result, the ellagic acid exhibits the effect of inhibiting an increase in a plasma endotoxin level, an increase in an intestinal TNF-α level, an increase in intestinal oxidative stress, a decrease in expression of intestinal TJ and AJ proteins, liver fat accumulation, an increase in plasma ALT, an increase in triglycerides, and hepatic oxidative stress and apoptosis. The composition containing ellagic acid as an active ingredient is provided as an intestinal microbe-improving composition and a preventive and therapeutic preparation for alcoholic fatty liver.

### CROSS REFERENCE TO RELATED APPLICATION

The present application claims priority to Korean Patent Application No. 10-2021-0019977, Feb. 15, 2021, the entire contents of which is incorporated herein for all purposes by this reference.

### BACKGROUND OF THE INVENTION

### 1. Field of the Invention

The present disclosure relates to an intestinal microbiome-improving composition including ellagic acid as an active ingredient.

### 2. Description of the Related Art

Alcohol is readily absorbed within the gastrointestinal tract. A portion of the absorbed alcohol is eliminated by the kidneys and lungs, and the rest is primarily detoxified by the liver. Chronic drinking causes imbalance of fat metabolism in hepatocytes through various pathways, induces alcoholic fatty liver, and causes alcoholic liver diseases. Conventionally, regarding the pathogenesis of alcoholic liver disease, it is known that ingested alcohol is converted to acetaldehyde by alcohol dehydrogenases (ADH) and cytochrome P450 2E1, thereby inducing fatty liver leading to various liver diseases. However, in a recent study, it was reported that intestinal bacteria and endotoxin significantly increased in patients with alcoholic liver disease, causing alcoholic liver damage.

On the other hand, it has been reported that the composition ratio of intestinal microbes is closely related to the immunity of the human body, and the distribution of intestinal microbes is also related to diseases occurring in secondary organs such as the liver, brain, and kidneys as well as the intestinal immunity. When the junction between intestinal epithelial cells is destroyed due to drinking and intestinal leakage occurs, a lot of LPS of gram-negative bacteria in the intestine moves through the portal vein. This activates Kupffer cells in the liver and increases inflammatory cytokines, resulting in severe liver damage.

### DOCUMENT OF RELATED ART

(Patent Document 1) Korean Patent No. 10-1940425 (Jan. 18, 2019)

(Patent Document 2) Korean Patent No. 10-2212606 (Feb. 5, 2021)

### SUMMARY OF THE INVENTION

The present disclosure is made to solve the problems occurring in the related art and an objective of the present disclosure is to provide an intestinal microbe-improving composition containing ellagic acid as an active ingredient. The ellagic acid improves the distribution of intestinal microbes by inhibiting the growth of harmful bacteria such as Verrucomicrobia and Bacteroidetes occurring due to alcohol intake and promoting the growth of beneficial bacteria such as Firmicutes. As a result, the intestinal microbe-improving composition exhibits the effect of inhibiting an increase in a plasma endotoxin level, an increase in an intestinal TNF-α level, intestinal oxidative stress, a decrease in expression of intestinal TJ and AJ proteins, liver fat accumulation, an increase in plasma ALT, an increase in triglycerides, and oxidative stress and apoptosis in the liver.

The present disclosure provides an intestinal microbe-improving composition containing ellagic acid as an active ingredient.

The present disclosure provides a pharmaceutical composition for prevention or treatment of alcoholic fatty liver, the pharmaceutical composition containing the intestinal microbe-improving composition.

The present disclosure provides a health functional food composition for preventing or treating alcoholic fatty liver, the food composition containing the intestinal microbe-improving composition.

According to the present disclosure, the ellagic acid inhibits the growth of harmful bacteria such as Verrucomicrobia and Bacteroidetes attributable to alcohol intake and increases the growth of beneficial bacteria such as Firmicutes, thereby controlling the distribution of intestinal microbes. As a result, the ellagic acid exhibits the effect of inhibiting an increase in a plasma endotoxin level, an increase in an intestinal TNF-α level, intestinal oxidative stress, a decrease in expression of intestinal TJ and AJ proteins, liver fat accumulation, an increase in plasma ALT, increase in triglycerides, and oxidative stress and apoptosis in the liver. The composition containing the ellagic acid active ingredient is provided as an intestinal microbe-improving composition and as a preventive and therapeutic preparation for alcoholic fatty liver.

### BRIEF DESCRIPTION OF THE DRAWINGS

FIG. 1 is an experimental plan for evaluating changes in appendix microbiota between an alcohol-exposed group and an ellagic acid (EA)-pretreated group;

FIG. 2 is a graph showing changes in the distribution of intestinal microbes at the Phylum level with respect to an alcohol-exposed group and an EA-pretreated group;

FIG. 3 is a graph showing averaged changes in the intestinal microbial distribution of FIG. 2;

FIG. 4 is a graph showing gene-level changes in the genes of Lactobacillus and Bacteroides with respect to an alcohol-exposed group and an EA-pretreated group;

FIG. 5 is a graph showing gene-level changes of Escherichia coli with respect to an alcohol-exposed group and an EA-pretreated group;

FIG. 6 is an H/E-stained image showing intestinal leak changes with respect to an alcohol-exposed group and an EA-pretreated group;

FIG. 7 is a graph showing changes in the level of plasma endotoxin with respect to an alcohol-exposed group and an EA-pretreated group;

FIG. 8 is a graph showing changes in the level of TNF-α with respect to an alcohol-exposed group and an EA-pretreated group;

FIG. 9 is an evaluation result for changes in intestinal oxidative stress with respect to an alcohol-exposed group and an EA-pretreated group;

FIG. 10 is an evaluation result for changes in expression of tight junction (TJ) and anchoring junction (AJ) proteins with respect to an alcohol-exposed group and an EA-pretreated group;

FIG. 11 is an evaluation result for changes in apoptosis of intestinal cells with respect to an alcohol-exposed group and an EA-pretreated group;

FIG. 12 is an H/E-stained image showing fat accumulation in the liver with respect to an alcohol-exposed group and an EA-pretreated group;

FIG. 13 is an Oil Red O-stained image showing intestinal fat accumulation in the liver with respect to an alcohol-exposed group and an EA-pretreated group;

FIG. 14 is an evaluation result for changes in the level of triglyceride (TG) with respect to an alcohol-exposed group and an EA-pretreated group;

FIG. 15 is an evaluation result for changes in the level of plasma ALT with respect to an alcohol-exposed group and an EA-pretreated group;

FIG. 16 is an evaluation result for changes in blood alcohol concentration (BAC) with respect to an alcohol-exposed group and an EA-pretreated group;

FIG. 17 is an evaluation result for changes in the level of expression of CYP2E1 proteins with respect to an alcohol-exposed group and an EA-pretreated group;

FIG. 18 is an evaluation result for changes in reactive oxygen species (ROS) with respect to an alcohol-exposed group and an EA-pretreated group;

FIG. 19 is an evaluation result for changes in hepatic oxidative stress markers with respect to an alcohol-exposed group and an EA-pretreated group;

FIG. 20 is an evaluation result for changes in liver apoptosis markers with respect to an alcohol-exposed group and an EA-pretreated group;

FIG. 21 is an evaluation result for changes in cleaved caspase-3 staining with respect to an alcohol-exposed group and an EA-pretreated group; and

FIG. 22 is a TUNEL analysis result with respect to an alcohol-exposed group and an EA-pretreated group.

### DESCRIPTION OF THE PREFERRED EMBODIMENTS

As the terms used in the embodiments of the present disclosure, general terms currently widely used are selected while considering functions in the present disclosure. However, the terms may vary depending on the intention of the ordinarily skilled person in the art, judicial precedents, and the emergence of new technologies, etc. In addition, in certain cases, there may be a term arbitrarily selected by the inventor(s), and the meaning thereof will be described in detail in the description of the present disclosure. Therefore, the term used in the present disclosure should not be defined on the basis of the name of a simple term but should be defined on the basis of the meaning of the term and the entire contents of the present disclosure.

In addition, unless otherwise defined, all terms including technical and scientific terms used herein have the same meaning as commonly understood by those who are ordinarily skilled in the art to which this invention belongs. It will be further understood that terms, such as those defined in commonly used dictionaries, should be interpreted as having a meaning that is consistent with their meaning in the context of the relevant art and the present disclosure, and should not be interpreted in an idealized or overly formal sense unless expressly so defined herein.

Numerical ranges are inclusive of the values defined in that range. Every maximum numerical limitation given throughout this specification includes all lower numerical limitations as if the lower numerical limitation were expressly written. Every minimum numerical limitation given throughout this specification includes all higher numerical limitations as if the higher numerical limitation were expressly written. All numerical limitations given throughout this specification will include all better numerical ranges within the broader numerical limits, as if the narrower numerical limitations were expressly written.

Hereinafter, the present disclosure will be described in detail.

The present disclosure provides an intestinal microbe-improving composition containing ellagic acid as an active ingredient.

The improvement of intestinal microbes means inhibition of the growth of Verrucomicrobia and Bacteroidetes, which are harmful bacteria caused by exposure to alcohol, and promotion of the growth of Firmicutes, which are beneficial bacteria.

The intestinal microbe-improving composition suppresses intestinal leakage by inhibiting the level of plasma endotoxin and the level of intestinal TNF-α caused by exposure to alcohol. The intestinal microbe-improving composition suppresses intestinal oxidative stress caused by exposure to alcohol and restores the decreased expression of intestinal tight junction (TJ) and anchoring junction (AJ) proteins caused by exposure to alcohol. In addition, the intestinal microbe-improving composition inhibits liver fat accumulation attributable to exposure to alcohol, the increases in plasma ALT and triglycerides levels, hepatic oxidative stress caused by exposure to alcohol, and liver apoptosis attributable to exposure to alcohol.

The present disclosure provides a pharmaceutical composition for prevention or treatment of alcoholic fatty liver, the pharmaceutical composition containing the intestinal microbe-improving composition.

The pharmaceutical composition of the present disclosure may be prepared in a unit dose form through formulation using a carrier and a common formulation method that can be readily performed by the ordinarily skilled person in the art to which the present invention pertains or internalized into a multi-dose container.

The carrier is a carrier commonly used in formulation, and examples of the carrier includes dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil but are not limited thereto. The pharmaceutical composition of the present disclosure may further contain a lubricant, a wetting agent, a sweetening agent, a flavoring agent, an emulsifying agent, a suspending agent, a preservative, and the like, in addition to the ingredient described above.

In the present disclosure, the content of an additive included in the pharmaceutical composition is not particularly limited and may be appropriately adjusted within the content range used for conventional formulation. The composition of the present disclosure may be administered orally or parenterally (for example, intravenously, subcutaneously, intraperitoneally, or topically) as desired. For oral administration, the composition may be formulated as tablets, troches, lozenges, aqueous suspensions, oily suspensions, prepared powders, granules, emulsions, hard capsules, soft capsules, syrups or elixirs, or the like.

The present disclosure provides a health functional food composition for preventing or treating alcoholic fatty liver, the food composition containing the intestinal microbe-improving composition.

The present disclosure can be used as a common food product. The food composition of the present disclosure can be used as a health functional food. The term “health functional food” means a food product manufactured and processed using raw materials or ingredients useful for the human body in accordance with the Republic of Korea Health Functional Food Act, and the term “functionality” refers to intake for the purpose of obtaining useful effects for health purposes such as controlling nutrients for the structure and function of the human body or obtaining physiological effects.

The food composition of the present disclosure may contain common food additives, and the suitability as the “food additive” is determined according to the standards for each item specified in the general rules and general test methods for a food additives code approved by the Republic of Korea Ministry of Food and Drug Safety, unless otherwise specified.

The items listed in the “Food Additives Code” include: for example, chemical compounds such as ketones, glycine, potassium citrate, nicotinic acid, and cinnamic acid; natural additives such as persimmon color, licorice extract, crystalline cellulose, high pigment, and guar gum; mixed preparations such as a sodium L-glutamate preparation, a noodle-added alkali agent, a preservative agent, and a tar color agent.

The food composition of the present disclosure may be manufactured and processed in the form of tablets, capsules, powders, granules, liquids, pills, and the like.

For example, among health functional foods in the form of capsules, hard capsules may be prepared by mixing the composition according to the present disclosure and additives such as excipients and filling conventional hard capsules with the mixture, and soft capsules may be prepared by manufactured by mixing the composition of the present disclosure and with additives such as excipients and filling capsule bases such as gelatin with the mixture. The soft capsule formulation may contain a plasticizer such as glycerin or sorbitol, a colorant, a preservative, and the like, if necessary.

The term definitions for the excipients, binders, disintegrants, lubricants, flavoring agents, and the like are described in documents known in the art, and those having the same or similar functions are used. The type of food is not particularly limited, and includes all health functional foods in the ordinary sense.

In the present disclosure, the term “prevention” refers to any act of inhibiting or delaying a disease by administering the composition according to the present disclosure. In the present disclosure, the term “treatment” refers to any action of improving or beneficially changing the symptoms of a disease by administering the composition according to the present disclosure. In the present disclosure, the term “improvement” refers to any action for improvement from a bad state of a disease by administering or ingesting the composition of the present disclosure to an individual.

Hereinafter, to help the understanding of the present disclosure, experimental examples and examples will be described in detail. However, the experimental examples and examples described below are only to provided for illustrative purposes, and thus the scope of the present disclosure is not limited to the experimental examples and examples described below. The experimental examples and examples are provided so that the present disclosure will be thorough and complete and will fully convey the concept of the present disclosure to those skilled in the art.

### Experimental Example Experimental Materials and Methods

Experimental examples described below are intended to provide experimental examples commonly applied to each example according to the present disclosure.

### 1. Reagents and Animal Models

Ellagic acid used in the present disclosure was purchased from Sigma Chemical Co., Ltd. (St. Louis, Mo., USA).

All animal testing procedures were performed in accordance with Andong National University's small animal testing guidelines, and were approved by the Andong National University Animal Care and Use Committee. All mice were housed where food and water were provided autonomously, and lighting was controlled (12-hour light/dark cycle). The ellagic acid was orally administered to 6-week-old female C57BL/6J mice at a daily dose of 60 mg/Kg as a physiologically and clinically relevant dose, and 200 mg/Kg silymarin was administered as a positive control. Water was orally administered to control mice. After the administration of ellagic acid for 14 days, alcohol or dextrose (control) was orally administered to some of the mice at a dose of 5 g/Kg, three times at 12-hour intervals. The mice were sacrificied 1 hour after the last administration.

### 2. Histological Analysis and Serum ALT Measurement

Portions of the largest liver lobes or small intestines from the mice pretreated with ellagic acid and from the controls were fixed in neutral formalin. Paraffin-fixed blocks of the formalin-fixed liver or small intestine tissues were stained with hematoxylin/eosin (H/E) and cut into 4 μm by KNU Core Laboratories. To observe fat accumulation, the fixed frozen liver samples were cut into 10 μm and stained with Oil Red O by KNU Core Labs. Plasma ALT levels of each mouse were measured using a standard endpoint colorimetric assay kit (Bio Vision, Milpitas, Calif.).

### 3. Endotoxin Analysis

Plasma endotoxin levels were measured using a commercial endpoint LAL Chromogenic Endotoxin Quantitation Kit manufactured by Thermo Fisher Scientific (Waltham, Mass.) and having a concentration range of 0.015 to 1.2 EU/mL.

### 4. Determination of Triglycerides in the Liver

The level of triglyceride (TG) in the liver was measured using a commercially available kit manufactured by Asan Co., Ltd. (Gimpo, South Korea).

### 5. Plasma ROS and Alcohol Levels

Levels of plasma ROS were visualized with 2′,7′-dichlorofluorescein diacetate (DCFH-DA) purchased from Thermo Fisher Scientific. After the DCFH-DA was incubated at 37° C. for 20 minutes, DCFH-DA fluorescence was measured. Plasma alcohol levels were measured using a commercially available kit manufactured by BioVision.

### 6. CYP2E1 Activity Analysis Liver solutions were each analyzed with an ELISA kit for CYP2E1, manufactured by Claud-clone corporation (Houston, Tex.), according to the manufacturer's instructions.

### 7. Enzyme-Linked Immunosorbent Assay (ELISA)

Liver solutions were analyzed using an ELISA kit for TNF-α, manufactured by Abcam (Cambridge, United Kingdom), according to the manufacturer's instructions. To use equal amounts of protein for the ELISA, protein concentration was measured with BCA reagent manufactured by BioRad (Hercules, Calif.). Duplicate samples (n=4/group) from each solution were used for ELISA.

### 8. Immunoblot Analysis

Portions of liver tissue and small intestine from each mouse were homogenized with RIPA buffer. Equally pooled protein in an amount of 50 ug collected from different mouse samples within the same group were separated by SDS/PAGE and transferred to nitrocellulose membranes. These membranes were incubated with polyclonal antibodies against CYP2E1 (1:5,000 dilution; Abcam), p-JNK (1:1,000 dilution; Cell Signaling), 3-NT (1:5,000 dilution; Abcam), iNOS (1:5,000 dilution; Abcam), cleaved caspase-3 (1:1,000 dilution; Cell Signaling), ZO-1 (1:5,000 dilution; Abcam), and claudin-1 (1:1,000 dilution; Thermo Fisher). Each mouse monoclonal antibody against JNK (1:1,000 dilution; Cell Signaling), Bax (1:1,000 dilution; Santa Cruz Biotechnology), or +-actin (1:10,000 dilution; Santa Cruz Biotechnology) was used to detect specific antigenic targets. After washing the nitrocellulose membranes three times with PBS at 10-minute intervals, horseradish peroxidase (HRP)-conjugated anti-rabbit or anti-mouse IgG (manufactured by Santa Cruz Biotechnology) was diluted at a ratio of 1:5,000 and used as a secondary antibody. Relative protein images were determined using the HRP-binding secondary antibody and an ECL substrate (Thermo Fishers). The intensity of immune response bands was quantified by density using ImageJ software (National Institutes of Health).

### 9. TUNEL Assay

Small intestine and liver specimens were fixed overnight in 10% buffered formalin and embedded in paraffin. ApopTag peroxidase from an in-situ apoptosis detection kit (Millipore, Billerica, Mass.) was used to identify apoptotic intestinal cells or hepatocytes through TUNEL assay.

### 10. Immunohistochemistry Analysis

Immunohistochemical (IHC) staining of cleaved caspase-3 was performed on cultured paraffin-fixed liver or small intestine slides, with a rabbit-specific HRP/DAB (ABC) detection IHC kit (Abcam) according to the manufacturer's instructions.

### 11. 16S Sequencing and Bioinformatics

Fecal samples were aseptically collected from the cecum of each mouse and frozen at −80° C. DNA was extracted using the Mag-Bind Universal Pathogen DNA Kit (Chunlab, Seoul, South Korea) according to the manufacturer's instructions. DNA sequencing of bacterial 16S rRNA in each fecal sample was performed at Chunlab (https://www.chunlab.com/).

### 12. Statistical Analysis

Data were analyzed using the SPSS 25.0 program (SPSS Inc., Chicago, Ill., USA), and the mean standard deviation was considered significant when p<0.05 by one-way ANOVA. Once significance was detected, a Turkey's HSD test as a post-hoc test was used to compare differences between groups.

### Example 1. Effect of Ellagic Acid on an Intestinal Microbiome Level

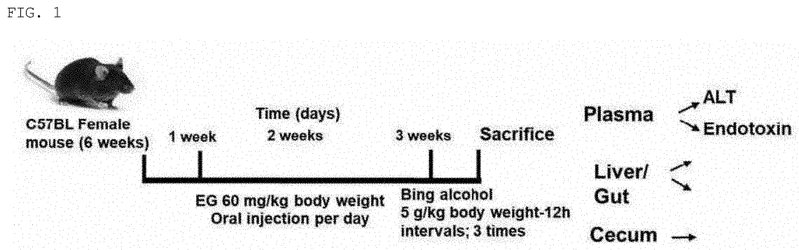

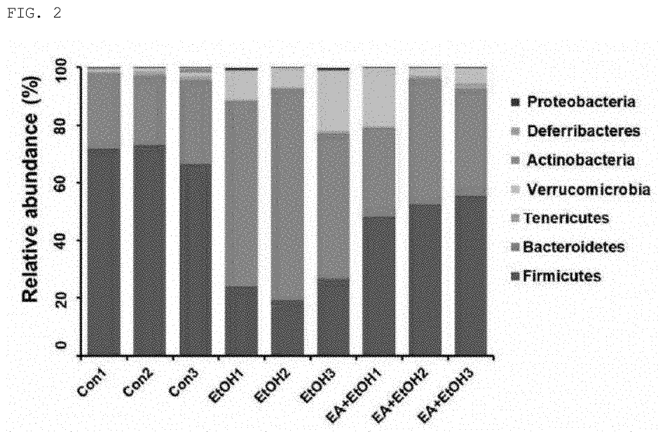

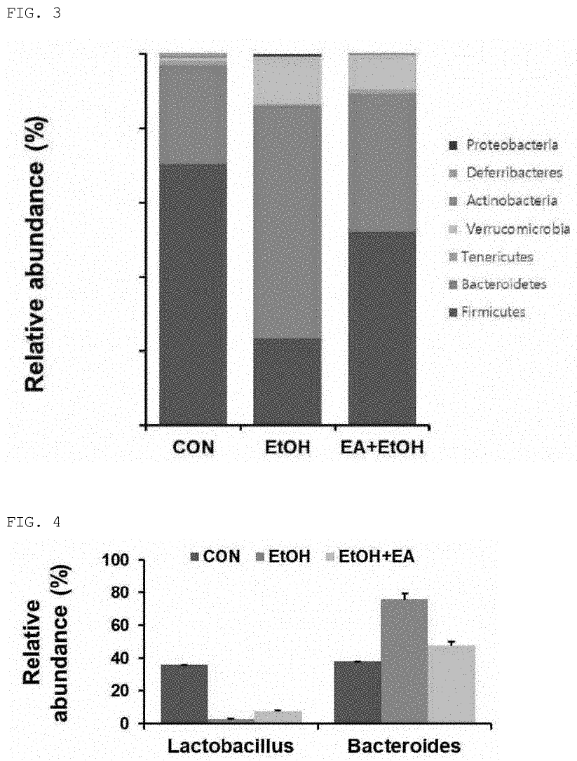

Intestinal microbiome and bacterial products have been associated with liver diseases (ALD). To evaluate the effect of ellagic acid (EA) on the intestinal microbial distribution in an alcoholic liver disease model, the cecal microbiota according to alcohol exposure and EA pretreatment in mice was evaluated according to the experimental design of FIG. 1. As shown in FIGS. 2 and 3, at the phylum level, there was no significant change in the intestinal microbial distribution of the control group and the EA pretreatment group. However, in the case of the alcohol exposure group, Verrucomicrobia phylum increased whereas Firmicutes phylum decreased. In addition, as shown in FIG. 4, the gene of the genus Bacteroides was found to be the most abundant in the alcohol-exposed group, but the gene of the genus Bacteroides was found to be decreased in the EA-pretreated mice. In addition, as shown in FIG. 5, the gene level of E. coli was increased in the alcohol-exposed group, but the gene level of E. coli was decreased to a level similar to that of the control group in the EA-pretreated mice.

### Example 2. Effect of Ellagic Acid on Plasma Endotoxin and Intestinal TNF-α

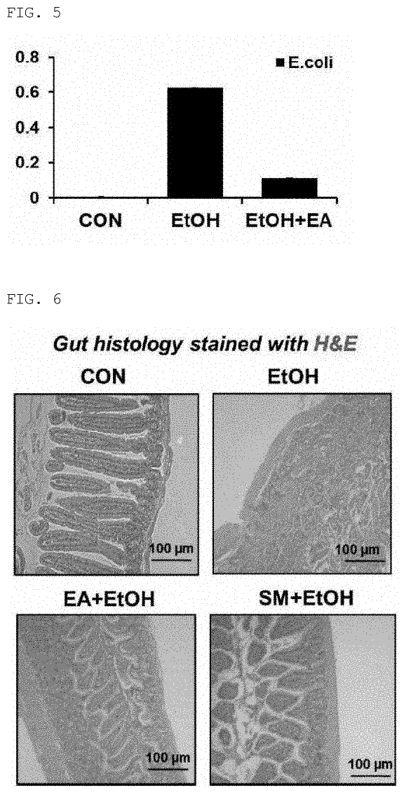

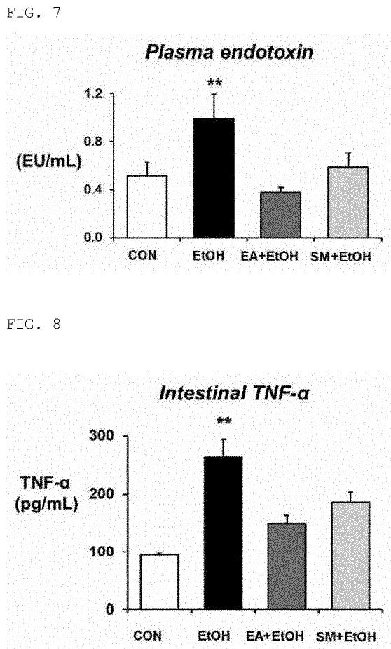

Intestinal microbial products can be stimulated by intestinal leak and endotoxins. Plasma endotoxin and intestinal TNF-α levels were measured to determine whether ellagic acid (EA)-mediated prophylaxis occurred at altered levels of microbial composition. As shown in FIG. 6, disintegration and detachment of many intestinal epithelial cells with abnormal morphology were observed in the alcohol-exposed group compared to the control group on the H/E-stained histological slides. In addition, as shown in FIG. 7, the alcohol-exposed group showed a significantly higher plasma endotoxin concentration than the control group, but this increase was suppressed in the EA-pretreated group. In addition, as shown in FIG. 8, the alcohol-exposed group showed an increased intestinal TNF-α level, but it was found that this increase was suppressed by EA pretreatment.

### Example 3. Effect of Ellagic Acid on Intestinal Oxidative Stress

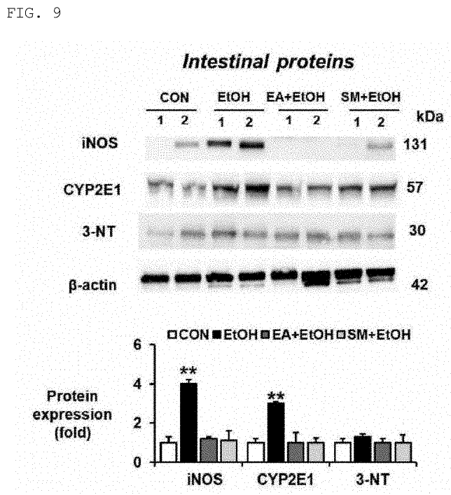

Oxidative stress, including levels of CYP2E1, iNOS, and nitridation proteins, is known to be a major factor of alcohol-mediated intestinal barrier dysfunction. To evaluate the effect of ellagic acid on intestinal oxidative stress, the expression levels of CYP2E1, iNOS, and 3-NT proteins were measured. As shown in FIG. 9, the levels of CYP2E1, iNOS, and 3-NT proteins in the intestine were significantly increased in the alcohol-exposed group, whereas the levels of CYP2E1, iNOS, and 3-NT proteins in the EA-pretreated group were decreased. The results demonstrate that EA pretreatment is effective in alleviating alcohol-mediated intestinal leakage by inhibiting the expression of proteins such as CYP2E1, iNOS, and 3-NT that cause intestinal oxidative stress.

### Example 4. Effect of Ellagic Acid on Intestinal Tight Junctions, Anchoring Junctions, and Apoptosis

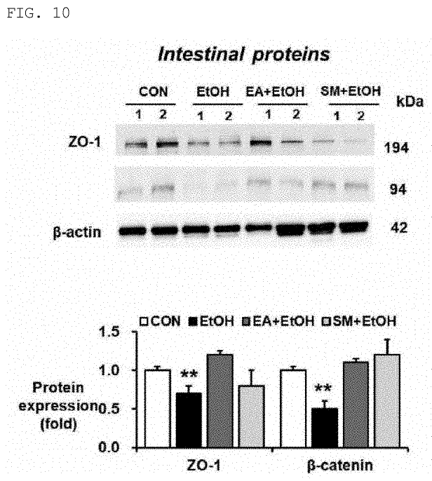

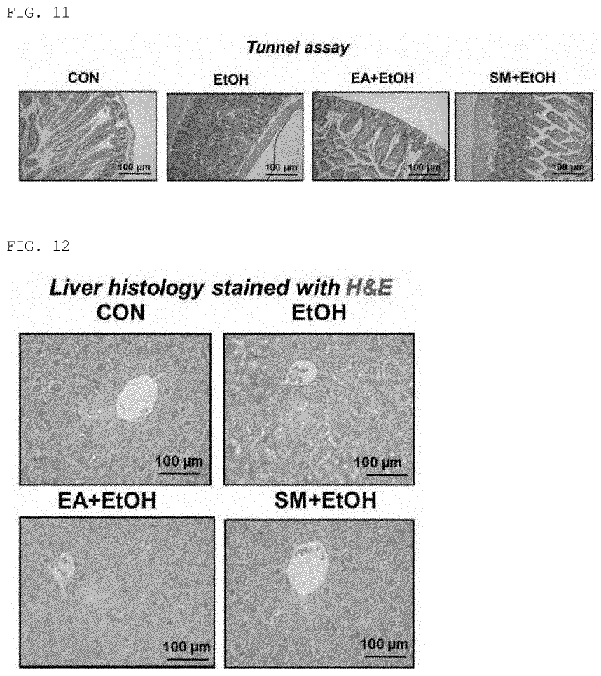

Changes in the expression of intestinal tight junction (TJ) and anchoring junction (AJ) proteins are observed in mouse models with alcohol-induced intestinal leak. To evaluate whether ellagic acid (EA) prevents alcohol-induced intestinal leakage, the expression levels of intestinal TJ and AJ proteins after the ellagic acid administration were measured. As shown in FIG. 10, the alcohol-exposed group showed a significant decrease in the levels of ZO-1 and β-catenin whereas the EA-pretreated group within the alcohol-exposed group showed recovery in the levels of ZO-1 and β-catenin. In addition, as shown in FIG. 11, the EA pretreatment group showed a decrease in apoptosis of intestinal cells that induce alcohol-induced intestinal death. These results demonstrate that administration of EA restores the levels of intestinal TJ and AJ proteins that increase intestinal apoptosis.

### Example 5. Effect of Ellagic Acid on Hepatic Fat Accumulation, Plasma ALT level, and Hepatic Triglyceride (TG) Level

Alcohol intake may induce intestinal leak and contribute to development in fatty liver, leading to alcoholic liver disease.

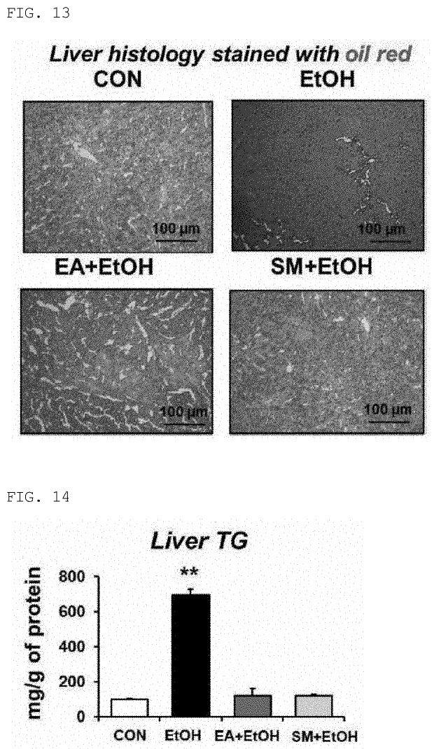

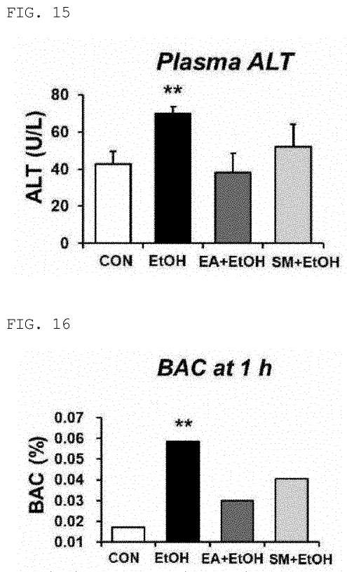

To evaluate the effect of ellagic acid (EA) on hepatic fat accumulation, and plasma ALT and hepatic triglyceride levels, liver tissues taken from mouse models were histologically analyzed through H/E staining and Oil Red O staining. As shown in FIGS. 12 and 13, it was found that the fat accumulation in the liver was significantly increased in the alcohol-exposed group, and the fat accumulation in the liver was decreased and the inflammatory foci was increased in the EA-pretreated group. In addition, as shown in FIGS. 14 and 15, the hepatic triglyceride (TG) level and the plasma ALT level were increased in the alcohol-exposed group, but the TG level and the plasma ALT level were decreased in the EA-pretreated group. In addition, as shown in FIG. 16, in the EA-pretreated group, the increase in blood alcohol concentration (BAC), caused by alcohol intake, was suppressed. The results demonstrate that ellagic acid (EA) has the effect of significantly reducing fat accumulation, and lowering plasma ALT and hepatic TG levels in a mouse model with alcohol-induced intestinal leakage.

### Example 6. Effect Of Ellagic Acid on Hepatic Oxidative Stress Marker

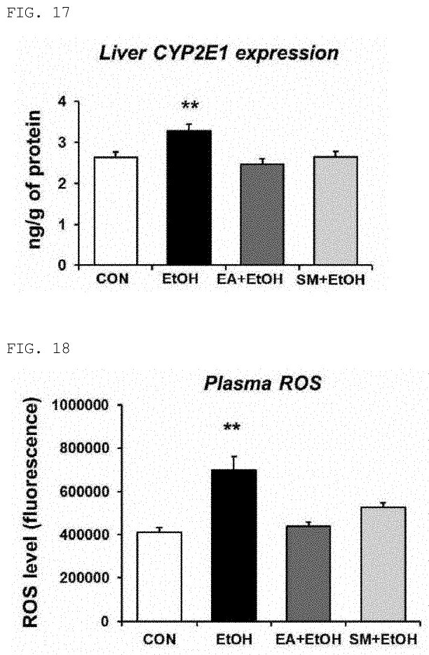

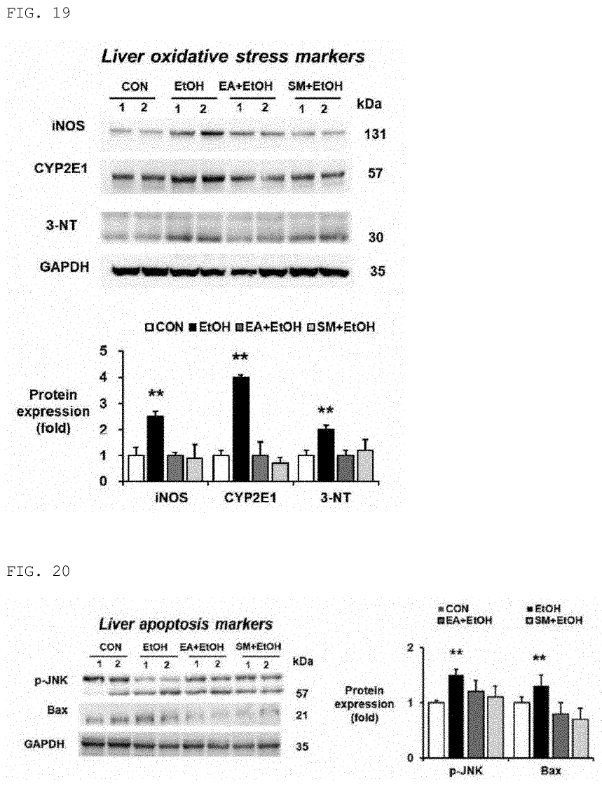

CYP2E1 plays an important role in alcohol-induced oxidative stress, contributing to alcohol-induced liver damage and intestinal leakage. To evaluate the effect of ellagic acid (EA) on hepatic oxidative stress, the expression and activity levels of CYP2E1 proteins were measured. As shown in FIGS. 17 and 19, the expression and activity levels of CYP2E1 proteins were significantly increased in the alcohol-exposed group, but the expression and activity levels of CYP2E1 proteins were decreased in the EA-pretreated group. Oxidative stress marker proteins such as inducible nitric oxide synthase (iNOS) and 3-NT proteins were significantly increased in alcohol-exposed mice but were decreased in the EA-pretreated group.

CYP2E1 generates reactive oxygen species (ROS) in alcoholic and nonalcoholic liver disease. As shown in FIG. 18, the level of plasma ROS was significantly increased in the mice exposed to alcohol, but an increase in the level of plasma ROS attributable to alcohol intake was suppressed. The above results demonstrate that EA has the effect of inhibiting the expression of hepatic oxidative stress marker proteins in alcohol-exposed mice.

### Example 7. Effect of Ellagic Acid on Liver Apoptosis Marker Protein

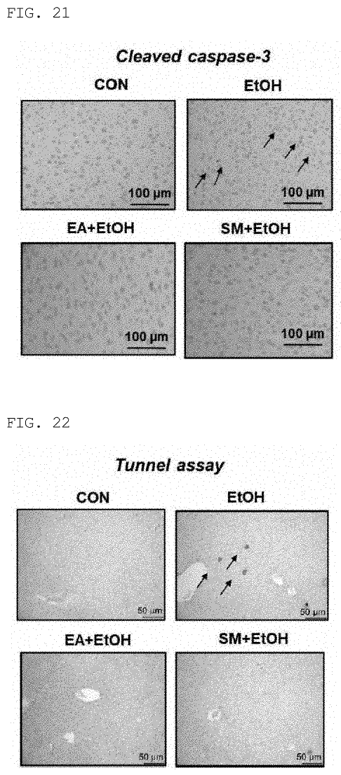

To determine whether ellagic acid (EA) reduces an increase in cell death in alcohol-exposed mice, Western blot analysis and cleaved caspase-3 analysis were performed. As shown in FIG. 20, p-JNK and Bax, which are apoptosis marker proteins, were increased in the alcohol-exposed group but were decreased in the EA-pretreated group. In addition, as shown in FIGS. 21 and 22, cleaved caspase-3 staining and tunnel analysis were easily observed in mice exposed to alcohol, and it was confirmed that hepatocyte apoptosis was increased. In contrast, the apoptosis marker proteins were decreased in the EA-pretreated group. The above results demonstrate that the administration of ellagic acid (EA) can suppress the increase in apoptosis marker proteins caused by alcohol.

As described above, a specific part of the present disclosure has been described in detail, and those who ordinarily skilled in the art will appreciate that the specific description is only a preferred embodiment and the scope of the present disclosure is not limited by the specific description. That is, the substantial scope of the present disclosure will be defined by the appended claims and their equivalents.

What is claimed is:

1. A method of preventing or treating alcoholic fatty liver, comprising:
administering a pharmaceutical composition comprising an intestinal microbiome-improving composition comprising ellagic acid as an active ingredient to a subject.

2. The method of claim 1, wherein the improvement of the intestinal microbiome refers to inhibiting growth of Verrucomicrobia and Bacteroidetes which are harmful bacteria caused by exposure to alcohol and promoting growth of Firmicutes that are beneficial bacteria.

3. The method of claim 1, wherein the intestinal microbiome-improving composition inhibits an increase in level of plasma endotoxin and an increase in level of intestinal TNF-α, attributable to exposure to alcohol, thereby suppressing intestinal leakage and improving intestinal microbes.

4. The method of claim 1, wherein the intestinal microbiome-improving composition inhibits intestinal oxidative stress caused by exposure to alcohol.

5. The method of claim 1, wherein the intestinal microbiome-improving composition enables expression of intestinal tight junction (TJ) and anchoring junction (AJ) proteins to be restored from a decrease caused by exposure to alcohol.

6. The method of claim 1, wherein the intestinal microbiome-improving composition inhibits liver fat accumulation and increased in levels of plasma ALT and triglycerides, attributable to exposure to alcohol.

7. The method of claim 1, wherein the intestinal microbiome-improving composition inhibits hepatic oxidative stress attributable to exposure to alcohol.

8. The method of claim 1, wherein the intestinal microbiome-improving composition inhibits liver apoptosis attributable to exposure to alcohol.

9. A method of preventing or treating alcoholic fatty liver, comprising:
administering a health functional food composition comprising an intestinal microbiome-improving composition comprising ellagic acid an active ingredient to a subject.

10. The method of claim 9, wherein the improvement of the intestinal microbiome refers to inhibiting growth of Verrucomicrobia and Bacteroidetes which are harmful bacteria caused by exposure to alcohol and promoting growth of Firmicutes that are beneficial bacteria.

11. The method of claim 9, wherein the intestinal microbiome-improving composition inhibits an increase in level of plasma endotoxin and an increase in level of intestinal TNF-α, attributable to exposure to alcohol, thereby suppressing intestinal leakage and improving intestinal microbes.

12. The method of claim 9, wherein the intestinal microbiome-improving composition inhibits intestinal oxidative stress caused by exposure to alcohol.

13. The method of claim 9, wherein the intestinal microbiome-improving composition enables expression of intestinal tight junction (TJ) and anchoring junction (AJ) proteins to be restored from a decrease caused by exposure to alcohol.

14. The method of claim 9, wherein the intestinal microbiome-improving composition inhibits liver fat accumulation and increased in levels of plasma ALT and triglycerides, attributable to exposure to alcohol.

15. The method of claim 9, wherein the intestinal microbiome-improving composition inhibits hepatic oxidative stress attributable to exposure to alcohol.

16. The method of claim 9, wherein the intestinal microbiome-improving composition inhibits liver apoptosis attributable to exposure to alcohol.
